(19) United States
(12) Patent Application Publication
(10) Pub. No.: US 2012/0149780 A1
(43) Pub. Date: Jun. 14, 2012

(54) Method of Use of Vitamin K as Energy Enhancer in Diverse Disease States

(76) Inventors: Dilip S Mehta, Mumbai (IN); Anselm De Souza, Mumbai (IN); Ashok B. Vaidya, Mumbai (IN); Rama A. Vaidya, Mumbai (IN); Dale Kriz, Phoenix, AZ (US); Kiran Krishnan, Bolingbrook, IL (US)

(21) Appl. No.: 13/382,618

(22) PCT Filed: Mar. 12, 2010
(86) PCT No.: PCT/IN10/00143

§ 371 Date: Jan. 6, 2012

(30) Foreign Application Priority Data
Mar. 12, 2009 (IN) 75/MUM/2009

(51) Int. Cl. A61K 31/122; A61P 3/00

(52) U.S. Cl. 514/681

(57) ABSTRACT

The invention relates to Vitamin K, its derivatives and combinations to increase the energy levels in diverse disease states and life style disorders, which are characterized by low energy level due to inadequate VO2ₘₐₓ, and pO₂ and low availability of ATP molecules. VO₂ₘₐₓ, peak oxygen uptake, is intimately connected to several diseases and life style disorders such as Metabolically Obese but Normal Weight (MONW), Overweight/Obese, diabetes mellitus, coronary artery disease, hypertension, cerebral vascular insufficiency, immune deficient states, cancer, aging-related disorders, reduced cardiopulmonary reserves and muscular fitness in athletics, high altitude climbing and exercise. The invention discloses that innovative blends of components that, in unique combination, synergistically bestow enhancement of VO₂ₘₐₓ leading to higher energy level, less fatigability and energy adaptations to stressful stimuli in humans and animals. Thus, vitamin K, its derivatives and combinations enhance the energy availability, primarily by the activation of AMP protein kinase (AMPK).

### FIELD OF INVENTION

This invention relates to the use of vitamin K or its derivatives, alone or in combinations thereof with other active agents to increase energy levels and ameliorate disease states and life style disorders.

### BACKGROUND OF INVENTION

The invention provides novel rationale and combinations for fundamental mechanisms operated by vitamin K. The rationale provides substratum for several of the broad spectrum of activities of vitamin K serendipitously observed clinically and claimed in PCT Application PCT/IN2008/000465 which is incorporated herein by reference in its entirety. Hypoxia and energy kinetics are intimately involved in ‘cardiorespiratory fitness’ with diverse disease states and life style disorders. Enhanced energy level and oxygen utilization are central to a functional capacity.

‘Cardiorespiratory fitness’ is an integrated concept of work capacity, based on energy level, which could be compromised by effect on VO₂ₘₐₓ, substrate utilization and ATP generation.

VO₂ₘₐₓ is “the highest rate of oxygen consumption attainable during maximal or exhaustive exercise”[1]. As exercise intensity increases so also oxygen consumption increases parallel until a point where exercise intensity can continue to increase as oxygen consumption plateaus; this point is considered the VO₂ₘₐₓ.

‘Cardiorespiratory fitness’ is interchangeably described as ‘aerobic fitness’ or ‘aerobic capacity’ or ‘physical fitness’ and more technically as ‘maximal oxygen uptake’ i.e. VO₂ₘₐₓ. Cardiorespiratory fitness refers to the ability of the circulatory and respiratory systems to supply oxygen to skeletal muscles during sustained physical activity.

Cardiorespiratory fitness improves the respiratory function by increasing the amount of oxygen that is inhaled and distributed to the body tissues, as well as its better tissue utilization and energy transduction.

Chronic and intermittent deficits in O₂ supply to the body, precipitates into a variety of dysfunctions. These deficits may result even from normoxic hypoxia (low levels of oxygen in the tissue) due to microcirculation defects just as much as due to hypoxemic (low level of oxygen in the blood) hypoxia. Effective therapy needs to bring about homeostatic mechanisms that elicit corrective changes in respiratory, circulatory and energy transduction function to maintain enhanced O₂ levels and/or improve the O₂ affinity of the mitochondrial enzyme system for adequate generation of ATP molecules.

In the cardiorespiratory diseases there is a low level of oxygen in tissues and the blood. In eukaryotic cells, oxygen is the major source for energy. Mitochondria are the sites of cellular respiration and convert energy, through oxidative phosphorylation, into forms that are usable by the cell (i.e.) to adenosine triphoshate (ATP). Nutrients from food sources (fats, carbohydrates and proteins) are enzymatically broken down to readily utilizable substrates in the cytoplasm, in the mitochondria matrix or in both places, to generate energy.

The entry of glucose into cell and utilization of free fatty acid and their subsequent catabolism generates ATP molecules. So, during oxidative phosphorylation, electrons are transferred from electron donors to electron acceptors in redox reactions. In eukaryotes, these redox reactions are carried out by a series of protein complexes within mitochondria where oxygen acts as terminal electron acceptor. These enzymes generate ATP from adenosine diphosphate (ADP) in a phosphorylation reaction. Therefore, if there is deficiency of oxygen at cellular level, and subsequent inefficient glucose utilization and oxidative phosphorylation will result in a decrease in ATP generation and thus resulting in lower availability of energy.

The present invention relates to use of vitamin K or its derivatives, alone or in combinations thereof with other active agents which improves oxidative phosphorylation through a novel mechanism. As in many bacterial species, menaquinones (MK) function as reversible redox component of the Electron Transfer Chain (ETC), mediating electron transfer between hydrogenases and cytochromes [2, 3]. It has been observed in B. cereus that the electron flow capacity increases simultaneously with the increase in menaquinone levels and vice versa [4]. Escamilla et al [4] also observed that during early germination, NADH-dependent respiration and cytochrome reduction were restored simultaneously with a four-fold increase in menaquinone content when compared with dormant state. This means that presence of menaquinones play an important role in the electron transport chain to support ATP generation during hypoxic conditions as electron acceptor in tandem with cytochromes in prokaryotes. A similar mechanism is postulated for eukaryotes in hypoxic environments i.e. in mitochondrial site of ATP generation.

One of the highly conserved metabolic pathway is that of AMP-activated kinase (AMPK) which is today recognized as a metabolic regulator of cellular enzyme involved in the generation of ATP molecules through carbohydrate and fat metabolism.

In itself AMPK activation is governed by the increase in AMP:ATP ratio. It is well known that exercise and metabolic stress tends to increase AMP:ATP ratio. In case of exercise or metabolic stress there is prevalence of hypoxia and also in the case of ischemia.

The upstream AMPK activating enzymes as a group are called AMPK kinase or AMPKK. AMPKK activates AMPK and in-turn AMP:ATP ratio decreases by switching off ATP-consuming anabolic pathways and switching on ATP-producing catabolic pathways

A novel understanding of the mechanism of enhanced ATP generation is through the increased AMPK activation. Inventors of the present application postulate that marked amount of mitochondrial superoxide generation in the presence of vitamin K and its metabolite menadione stimulates AMPK, its upstream enzymes AMP-Activating Protein Kinase (AMPKK) and as yet undiscovered but proposed synergistic pathways to improve ATP generation. All this will result in the enhanced utilization of oxygen for energy transduction.

VO₂ₘₐₓ is routinely used to measure the cardiovascular output of athletes to assess their sports performance, of various age groups to prevent life-style related diseases (LSRD) and in many disease states to determine treatment modality.

Epidemiologic and clinical evidence demonstrate that a poor cardiorespiratory fitness is a major risk factor for life-style related diseases (LSRD) such as obesity, hypertension, hypercholesterolemia, arteriosclerosis and diabetes [5-8]. Moreover, low cardiorespiratory fitness has been found to be a predictor of cardiovascular disease (CVD) mortality, and all-cause mortality [9-12]. Thus, it is essential to maintain a high level of cardiorespiratory fitness to prevent LSRD.

Since the time of WHO Ottawa Charter of 1986 [13], a revolution has been occurring in the field of health promotion and the priorities for action needed to facilitate health for all people. The position papers disseminated by the World Health Organization (WHO) Europe Health Promotion Office, and furthered by the Ottawa Charter, the Environmentally Preferable Purchasing (EPP) Report in Canada, the Healthy Cities project, as well as other efforts, have triggered the new health promotion movement which has introduced new concepts about what constitutes health and how health promotion efforts should be configured to achieve health [14]. As a consequence, Industrial Safety and Health Law were revised in Japan in 1988. It prescribes that execution of health promotion measures for workers is an obligation of employers. In line with this, Total Health Promotion Plan (THP) was instituted [15]. THP includes not only medical examination, but also investigation of life-style and assessment of physical fitness. Determination of the maximal oxygen uptake (VO₂ₘₐₓ) is an indication of whole body endurance and is included in physical fitness assessment. Based on these results, guidance for health care and psychological counseling is provided to employees. It is reported that VO₂ₘₐₓ is influenced by life-style, such as exercise and smoking [8, 16-22]. Moreover, the preceding researches reported that the fall in VO₂ₘₐₓ is closely connected with cardiovascular system disorders, such as heart diseases, and hypertension [8, 9, 11, 12, 23-25]. Based on this evidence and from a viewpoint of preventive medicine, VO₂ₘₐₓ is being used as a health index in THP. Cardiovascular fitness is usually evaluated as the maximal oxygen uptake per body mass (VO₂ₘₐₓ mL·kg⁻¹·min⁻¹). The Japanese Ministry of Health Labor and Welfare in 2006 proposed VO₂ₘₐₓ reference values for each age group to prevent LSRD [26]. These VO₂ₘₐₓ reference values are referenced in the “Exercise and Physical Activity Reference Quantity for Health Promotion 2006 (EPAR2006)”. Original target, 15 years ago, was the prevention of coronary artery disease. However, following the establishment of this standard, the morbidity pattern of people has worsened and LSRD have increased in prevalence. In order to face this situation the EPAR2006 was made based on the latest scientific evidence and was designed to maintain and promote the health of people and prevent LSRD by improving their capacity for physical activity and exercise. These VO₂ₘₐₓ reference values proposed in the EPAR2006 were determined by experts through the systematic review of literature regarding the relationship between VO₂ₘₐₓ and LSRD such as obesity, hypertension, hypercholesterolemia, diabetes, cerebrovascular disease, CVD mortality and all-cause mortality.

VO₂ₘₐₓ decreases with age. The average rate of decline is generally accepted to be about 1% per year or 10% per decade after the age of 25. One large cross sectional study found the average decrease was 0.46 ml/kg/min per year in men (1.2%) and 0.54 ml/kg/min in women (1.7%) [27, 28].

Thus it becomes clear that any increase in VO₂ₘₐₓ is an increase in health level which is useful in resisting fatigue, susceptibility to stress and infection and reduction in cardiovascular fitness. It is very important to realize that higher VO₂ₘₐₓ when enhanced by physical training has underlying biochemical and molecular mechanisms. The latter can be targeted through dietary supplements etc, even without physical training. ‘Exercise in a pill’ has been reported by Narkar et al [29] suggesting that without any exercise, you can take a drug and chemically mimic it. Their earlier work reported, in a presentation at the American Society for Biochemistry and Molecular Biology, that administration of a synthetic drug 5-aminoimidazole-4-carboxamide ribonucleoside (AICAR) is now able to chemically switch on (peroxisome proliferator-activated receptor δ), PPAR-δ, the master regulator that controls the ability of cells to burn fat [30].

Several such attempts have been made, mostly to improve athletic performance. Many supplemental trials to improve VO₂ₘₐₓ have shown poor or no improvement at all. Some of them are:

1. Betaine [31]. 2. Oral phosphate loading in healthy individuals produced no improvement in aerobic capacity [32]. 3. Tricarboxylic-acid-cycle intermediates (TCAIs; pyridoxine-alpha-ketoglutarate, malate, and succinate) and other substances potentially supporting the TCA cycle (such as aspartate and glutamate) does not improve cycling performance [33]. 4. Supplementation with neither vitamin E nor C, either alone or in combination, enhanced exercise performance [34]. 5. Exercise performance is unaffected by oral supplementation with lactate [35]. 6. Supplementation of American ginseng for 4 week prior to an exhaustive aerobic treadmill running did not enhance aerobic work capacity [36]. 7. L-carnitine is unlikely to be associated with the enhanced exercise performance [37]. 8. Inosine supplementation has no effect on aerobic performance [38]. 9. Nicotinamide Adenine Dinucleotide Hydride (NADH) supplementation of 30 mg per day, in a 4 weeks' duration, balanced, double-blind, and cross-over design, had no effects on maximal oxygen uptake VO₂ₘₐₓ, maximal anaerobic running time or mental performance [39]. 10. The supplementation of chronic consumption of medium-chain triacylglycerols (MCT) neither enhances endurance nor significantly alters performance-related metabolism in trained male runners [40]. 11. Oral creatine supplementation improves exercise performance in competitive squash players [41]. 12. Creatine monohydrate supplementation did not increase performance [42].

U.S. Pat. No. 6,117,872 [43] claims administration of high levels of basic amino acids to improve physical capacity of individuals. Amino acids of choice are those that enhance vascular nitric oxide synthesis or activity to cause vasodilatation of vessels supplying exercising skeletal muscles and thereby enhancing aerobic capacity.

U.S. Pat. No. 7,071,183 [44] uses inhibitors of rennin-angiotensin system to prevent conditions associated with hypoxia or impaired metabolic function or efficiency and may be used to enhance function in healthy subjects. Whereas, U.S. Pat. No. 6,368,640 [45] provides use of a dietary supplement comprising L-arginine, alone or in combination with ginseng and Gingko biloba with other nutritional supplements to bestow sexual wellness upon a human when taken as a dietary supplement.

These various patented approaches have, in fact, been aimed at improving energy levels and thus also improved oxygen utilization.

A 1973 published study showed that in previously sedentary people, training at 75% of aerobic power, for 30 minutes, 3 times a week over 6 months increases VO₂ₘₐₓ an average of 15-20% [46]. It seems that training can slow the rate of decline in VO₂ₘₐₓ but becomes less effective after the age of about 50[47]. Recently the biochemical mechanisms leading to beneficial effects of exercise are being explored for development of new energizers. This research is being described as an “Exercise in a Pill” [29, 30].

The present invention claims supplementation of vitamin K or its derivatives, alone or in combinations there of with other active agents which enhances energy transduction processes, VO₂ₘₐₓ and cardiorespiratory fitness.

### SUMMARY OF THE INVENTION

The present invention relates to the use of vitamin K or its derivatives, alone or in combinations thereof with other active agents to increase energy levels and ameliorate disease states and life style disorders.

In one aspect, the invention provides a method of alleviating hypoxemia and hypoxia, resulting in increased energy levels comprising administering to a mammal a composition of vitamin K or its derivatives alone or in combination with other active agents.

In this method, Vitamin K or its derivatives activate AMPK and is associated with (a) restoration of energy in chronic fatigue syndrome (b) countering the post-ischemic effects in heart, brain, muscles, liver and kidneys (c) increase in life-span in caloric restrictions (d) improved quality of life in patients with cancer (e) significant amelioration of metabolic syndrome (f) reduction in inflammatory cytokines and improved adipose endocrine harmony (g) relief of lipid deposition in arteries and liver and (h) increased tumor suppression.

In another aspect, the invention provides a method of improving tissue perfusion, increasing VO₂ₘₐₓ and activation of AMPK by administering to a mammal a composition of vitamin K or its derivatives, a combination of vitamin K or its derivatives alone or in a combination thereof with other active agents. Increase in VO₂ₘₐₓ influences the technical and tactical performance in all sports.

In still another aspect, the invention provides a method to maintain a balanced homeostatic state in geriatric population by improvement in tissue perfusion and activation of AMPK comprising administering to the said population a composition of vitamin K or its derivatives, alone or in combination thereof with other active agents.

In yet another aspect, the invention provides a method of ameliorating the condition of MONW in a mammalian subject by administering a composition of vitamin K or its derivatives, alone or in combination thereof with other active agents.

In further aspect, the invention provides a method to improve the quality of life in cancer patients by activation of AMPK comprising administering vitamin K or its derivatives, alone or in combination thereof with other active agents.

In a different aspect, the invention provides a method to improve the outcome in postoperative cardiopulmonary complications resulting from surgical lung resection in lung cancer patients comprising supplementing a composition of vitamin K or its derivatives, a combination of vitamin K or its derivatives thereof alone or in combination thereof with other active agents.

In still different aspect, the invention provides a method to lower significant risk factors of the physical work capacity (VO₂ₘₐₓ) in diabetic patients and their first degree relatives by supplementing vitamin K or its derivatives, a combination of vitamin K or its derivatives, alone or in combination thereof with other active agents.

In yet another aspect, the invention provides a method to reduce acanthosis nigricans and hyperpigmentation through supplementation of a composition of vitamin K or its derivatives, a combination of vitamin K or its derivatives alone or in combination thereof with other active agents.

In a further aspect, the invention provides a method to lower inflammatory C-reactive protein (CRP) marker by supplementing a composition of vitamin K or its derivatives, alone or in combination thereof with other active agents. In this method, the reduction in inflammatory CRP marker is associated with reduction in atherosclerosis and thereby reduction in hypertension.

Also provided by the invention is a method of reducing normoxic hypoxia and hypoxemic hypoxia, which brings about homeostatic mechanism and maintains enhanced O₂ levels and/or improve the O₂ affinity of the mitochondrial enzyme system for adequate generation of ATP molecules, comprising supplementing a composition of vitamin K or its derivatives, alone or in combination thereof with other active agents.

### DESCRIPTION OF THE INVENTION

The broad spectrum effects of vitamin K suggested that there may be one or more underlying basic mechanisms of its action. The improvement in antihypoxic function of tissues would lead to enhanced energy transduction. The fundamental mechanism for energy transduction is conventionally considered to be maximal oxygen uptake viz. VO₂ₘₐₓ.

The serendipitous clinical findings of increased energy level, less fatigue and increased endurance of muscular exertion, after administration of vitamin K2-7, suggests that there may be an important target of the vitamin, besides the known carboxylation of Glu to Gla proteins. Marked amounts of O₂⁻ (Superoxide free radical) have been reported as a consequence of the metabolism of menadione, both in guinea pig and in rat heart [48]. Menadione is a vitamin K metabolite [49]. In Staphylococcus aureus, auxotrophic for menadione, the deprivation of the latter causes a decrease in cellular levels of cytochromes, protoheme, vitamin K2 and several membrane bound flavoprotein dehydrogenases [50]. The cytochromes are maintained in the same proportion as menadione-supplemented cells. In spite of that, oxidative phosphorylation was reduced tenfold. But ATP hydrolysis is not affected. These data suggest an important role of menadione and vitamin K2 in the electron transport chain and free radical generation [50]. The post-ischemic generation of free radicals can be substantial and harmful to the cells. A flurry of superoxide flashes occurring during reoxygenation of cardiac muscle cells can be markers of diverse diseases due to oxidative stress and hypoxia etc. [51].

Vitamin K group counters these harmful effects through its diverse actions on AMP activated protein kinase (AMPK). These actions are proposed innovatively as follows:
1. Free radical—oxy and hydroxy activating AMPK [52, 53].
2. Thrombin of endothelial cells and AMPK [54]
3. Direct activation of AMPK by vitamin K group as is known with natural products—berberine [55], resveratrol [56] and (−) Epigallocatechin 3-Gallate [57].

AMP-activated protein kinase is a highly conserved sensor of cellular energy kinetics [58]. The energy homeostasis is regulated by the enzyme, whenever metabolic stress upsets it, for example in hypoxia, hyperglycemia, etc. the latter interfere with ATP synthesis. AMPK shifts the homeostasis to higher ATP generation through the catabolic pathway. Several enzymes get influenced by AMPK, which enhance energy level. The applicants propose a novel and non obvious mechanism wherein vitamin K group activates AMPK, the energy sensor. This leads to less fatigue, higher energy level and increased endurance of muscular exertion. It is claimed that vitamin K group can also serve as exercise mimetic through its effect on AMPK, which downstream works on PPARδ receptors as shown for an antagonist AICAR [29]. The clinical utility of exercise mimetic will be immense, with training or with other agents in low energy status due to diverse causes.

The benefits which accrue due to the novel use of vitamin K group is due to the results of activation of AMPK which includes but not limited to (1) Restoration of energy in chronic fatigue syndrome (2) Countering the post-ischemic effects in heart, brain, muscles, liver, kidneys etc. (3) Increase in life-span in caloric restrictions (4) Improved quality of life in patients with cancer (5) Significant amelioration of metabolic syndrome. (6) Reduction in inflammatory cytokines and improved adipose endocrine harmony (7) Relief of lipid deposition in arteries and liver and (8) Increased tumor suppression.

### Vitamin K and Sports Medicine

As several of the agents which enhance sports performance and endurance are drugs of abuse and doping has emerged as major challenge in competitive National and International sports. Hence, there is an urgent need to investigate and evolve non-addictive diet like substances which would enhance the excellence in sports without jeopardizing human health. This unmet need has been partly addressed in the present disclosure.

In otherwise healthy individual tissue hypoxia exists during endurance exercise. Hence any increase in VO₂ and hypoxia are really two sides of a coin. It is convenient to determine VO₂ₘₐₓ. This is reflected in many studies referred in this document where VO₂ₘₐₓ is co-related to a variety of disease states and athletic performances.

Mortensen et al [59] examined the regulatory limits of systemic and muscle perfusion in exercising humans, and concluded, that the locomotor skeletal muscle perfusion is restricted during maximal and supramaximal whole-body exercise in association with a plateau in cardiac output and limb vascular conductance. This means that there is decrease in O₂ concentration within the tissues which hampers the energy production.

Soccer players, during a soccer game, produce average exercise intensity of ˜75% to 85% of VO_{2max [}60-64]. Helgerud et al [62] showed that an improvement in VO₂ₘₐₓ of 18 ml/kg 0.75/min and 7% reduced energy cost of sub maximal running (i.e.) improved running economy—increased both the distance covered in a game by 1800 m and the average exercise game intensity by 4% [61]. Helgerud et al [62], further showed that increased aerobic capacity is associated with 24% more involvement with the ball and a doubling of the number of sprints performed. It is recognized that aerobic capacity consists of not only VO₂ₘₐₓ but also anaerobic threshold and running economy. However, Hoff et al [63] estimate that only a small part of the game is spent at the actual intensity corresponding to anaerobic threshold.

The physical demands upon players in professional tennis have been increasing over the past few years. Tennis is a sport based on short explosive bursts of near maximal intensity (5-10 s). Resting period may vary from 10 to 20 seconds. A match may last less than one hour to as long as five hours [65, 66]. Considering partial regeneration between points as well as between matches and tournaments a high cardiorespiratory capacity may help to avoid fatigue and aid in recovery. Roetert et al [67] suggest that technical and tactical skills, psychological preparation, game strategy, motor skills such as power, strength, agility, speed, and explosiveness, and a highly developed neuromuscular coordinating ability are strongly correlated with tennis tournament performance. Nevertheless, a major determinant of the outcome of the modern game in tennis is the player's aerobic fitness (VO₂ₘₐₓ), which not only enables the player to repeatedly generate explosive strokes and complete rapid on-court movements but also ensures fast recovery and contributes to maintaining concentration and preparation for the next rally during extended play [68-70].

Furthermore activation of AMPK during exercise has been shown in various studies in the past [7]-73]. The present invention claims that vitamin K or its derivatives, alone or in combinations there of with other active agents increase activation of AMPK through a novel pathway described earlier.

The applicants claim that vitamin K or its derivatives, alone or in combinations there of with other active agents play an important role in sports medicine including but not limited to modern soccer, tennis and most other sports and has a major influence on technical performance and tactical choices. This claim is based on the fact that vitamin K or its derivatives, alone or in combinations there of with other active agents improve the tissue perfusion, increases VO₂ₘₐₓ and enhances the activation of AMPK.

### Vitamin K and its Derivatives and Geriatrics

There is a great concern for the older adult health, functional capacity, quality of life, and independence as by the year 2030 the number of individuals 65 yr and over will reach 70 million in the United States alone; persons 85 yr and older will be the fastest growing segment of the population [74]. Paterson et al [75] state that there is a linear decline of VO₂ₘₐₓ and functional capacity from age 30 to 65 for various measures and a greater declining rate from the age 65 onwards. However, even up to the age of 85 the ability to sustain a relatively high intensity of aerobic exercise appears preserved.

Decline and deficiency in estrogen hormone in women at menopause and during post menopausal years expose them to loss of bone mass (osteopenia/osteoporosis), weight gain and waist gain with increase in body fat percent and sarcopenia. It has been said that menopausal state is equated with metabolic syndrome. Similarly, in men andropause is characterized by increase in central obesity, sarcopenia and vulnerability for coronary artery disease. Growth hormone deficiency and low level of testosterone are implicated in this condition. Fatigue and weakness expressed as ‘low energy level’ are common complaint in menopausal women [76, 77].

Aging is a complex process but it is certain that participation in regular physical activity elicits various favorable responses that contribute to healthy aging. Aging manifests itself by decline in a number of organ functions. Participation in regular physical activity is an effective intervention. Older population adapts well to both endurance and strength exercises. One of the markers of an improved cardiovascular function is as measured by maximal VO₂ₘₐₓ. But on the other hand an improved VO₂ₘₐₓ also means a greater ability in the aerobic and endurance exercise program.

Huonker et al [78] state that, with the beginning of the 4^{th} to 5^{th} decade of life there is decline in the number and size of the mitochondria. Consequently the aerobic muscular energy metabolism decreases by 8-10% per decade. Hence, any nutrient that improves mitochondrial energy transduction capacity will aid geriatric population.

The instant invention claims that supplementation of vitamin K or its derivatives, alone or in combinations there of with other active agents, with or without aerobic training, enhance VO₂ₘₐₓ and thus contribute to improved health status and an increase in life expectancy in the older adult population. Such a supplement may also provide a number of psychological benefits related to preserved cognitive function, alleviation of depression symptoms and behavior, and an improved concept of personal control and self-efficacy [74].

Inventors also claim that by improvement in tissue perfusion and the activation of AMPK, can lead to the balanced homeostatic state in geriatric population.

### MONW (Metabolically Obese, Normal Weight)

Individuals exist who are not obese on the basis of height and weight, but who, like people with overt obesity, are hyperinsulinemic, insulin-resistant, and predisposed to type 2 diabetes, hypertriglyceridemia, and premature coronary heart disease. Such individuals are called as Metabolically Obese, Normal-Weight (MONW) individuals [79, 80]. The Third Report of the National Cholesterol Education Program Expert Panel (Adult Treatment Panel III) not only draws attention to the importance of the metabolic syndrome but also provides the first practical definition of this syndrome [81]. St-Onge et al propose that ‘individuals whose BMI is within the normal to slightly elevated range, 18.5-26.9 kg/m², but who also fulfill the criteria for the metabolic syndrome be classified as MONW' [82].

People with the metabolic syndrome are at increased risk for developing diabetes mellitus [83] and cardiovascular disease [84] as well as increased mortality from cardiovascular disease and all causes [85]. Ford et al [86] carried out a survey to estimate the prevalence of the metabolic syndrome in the United States as defined by the ATP III report [81]. Their survey reported ˜24% of the US population had the metabolic syndrome.

One of the specific factors that appear to predispose MONW, as well as more obese individuals, is a low VO₂ₘₐₓ inclusive of insulin resistance, central fat distribution and inactivity. MONW individuals are frequently undetected and undiagnosed because of their normal BMI and young age. So there is need to identify MONW individuals and treating them with diet, exercise, and possibly pharmacological agents before these diseases become overt, or at least early after their onset.

Conus et al [87] noted that MONW women were less aerobically fit, expend fewer calories in their physical activity periods, and spent a greater portion of their time watching television. He further mentioned that, these types of biological attributes and behaviors likely contribute to the positive energy balance that leads to greater adiposity and higher total cholesterol among MONW women.

A logical next step, in terms of treatment, would be to examine the effects of mild caloric restriction and/or exercise programs to improve the metabolic profile of MONW women. The inventors have found that use of vitamin K or its derivatives, alone or in combinations there of with other active agents and vitamin K based formulations will improve the VO₂ₘₐₓ which is one of the predisposing factors in MONW and better energy transduction through AMPK activation.

By improving VO₂ₘₐₓ one enhances the ability to consume more oxygen and thus help improve the energy production, reduction in calories and ability to supply more oxygen to the cells.

The applicants in the instant disclosure claim that vitamin K or its derivatives, alone or in combinations there of with other active agents improve the tissue perfusion increase VO₂ₘₐₓ and enhance the activation of AMPK and thus helping in ameliorating the condition of MONR.

### Vitamin K and Quality of Life (QOL) in Cancer

Lung cancer patient's only curative treatment is lung resection. While there is an improvement in the outcome from lung resection over the years, there is still a high rate of morbidity and mortality as a result of postoperative cardiopulmonary complications [88-90]. Out of the many postoperative cardiopulmonary complications one that has been proposed to be the best predictor of the complications after surgical resection is exercise capacity expressed as maximal oxygen consumption (VO₂ₘₐₓ).

Benzo et al [91] concluded, through a meta-analytical approach, that exercise capacity expressed as VO₂ₘₐₓ is lower in patients that develop clinically relevant complications after curative lung resection. Gaballo et al [92] state that if the maximal oxygen uptake (VO₂ₘₐₓ) is greater than 15 ml/Kg, surgery can be performed, if VO₂ₘₐₓ is less than 15 ml/Kg, patients are inoperable. Hypoxia, i.e. low oxygen availability, induces changes in transcriptional regulation that serve to alter cellular metabolism (i.e., shift to glycolytic pathways) and promote the in-growth of immature, architecturally deranged, and highly permeable blood vessels that facilitate the passage of tumor cells into the circulation [93-96]. Thus, cancer's response to hypoxia not only sustains tumor growth and survival, but through angiogenesis it fosters invasion and metastasis. Indeed, hypoxic tumors have been reported to have a predilection for tissue invasion and metastasis [97, 98]. Such adaptability has been proposed to be especially relevant in pancreatic cancer, a malignancy that exhibits a profound and characteristic avascular, hypodense appearance on contrast-enhanced computerized tomography of the abdomen [99]. Therefore there is a need to improve oxygen availability.

A common consequence of cancer and its treatment is fatigue. Aside from the psychological factors, the main physiological factors leading to fatigue in cancer patients is anemia. This lowers VO₂ₘₐₓ, which is strongly related to circulating hemoglobin. Researchers have shown that aerobic exercise training has been demonstrated to greatly relieve symptoms of fatigue in patients with cancer [100]. Schmitz et al [101] have reviewed 32 studies of controlled trials of physical activity interventions in cancer survivors during and after treatment. They conclude that physical activity improves cardiorespiratory fitness (i.e. VO₂ₘₐₓ) during and after cancer treatment.

Sawada et al [102], researching at the Tokyo Gas Health Promotion Center, studied a cohort of 9039 men (19-59 yr) who were followed for mortality between 1982 and 1988 to 1999. They concluded that low cardiorespiratory fitness (low VO₂ₘₐₓ) is associated with cancer mortality in Japanese men. Inventors of current application claim that supplementation of vitamin K or its derivatives, alone or in combinations there of with other active agents assist, with or without aerobic training, enhance VO₂ₘₐₓ and thus contribute to improved health status.

It is known that AMPK plays a restrictive role in the growth and/or survival of some cancer cells [103]. Biochemical Journal reporting research at Alessi laboratory [103] shows that AMPK activators delay the growth of tumours that occur spontaneously in PTEN (phosphatase and tensin homologue deleted on chromosome 10) heterozygous mice. The study demonstrated that the LKB1 tumour suppressor phosphorylates and activates AMPK when cellular energy levels are low, thereby suppressing tumor growth. It also suggested that mammalian homolog of target of rapamycin (mTOR), which has been implicated in the pathogenesis of insulin resistance and many types of cancer, is inhibited by AMPK.

Papandreou et al [104] reported that oxygen deprivation i.e. hypoxia can activate the autophagic pathway in human cancer cell lines. Hypoxia-induced autophagy involved the activity of AMPK. They suggested that the autophagic degradation of cellular macromolecules contributes to the energetic balance governed by AMPK, and that suppression of autophagy in transformed cells can increase both resistance to hypoxic stress and tumorigenicity. As discovered by the inventor's vitamin K supports energy transduction through multiple pathways.

Inventors of current application claims pre-post supplementation of vitamin K or its derivatives, alone or in combinations there of with other active agents helps improve outcomes in postoperative cardiopulmonary complications faced by an inevitable curative surgical lung resection for lung cancer patients and for cancer patients in general through improvements in tissue perfusion thus providing sufficient oxygen concentration and reliving hypoxia and hypoxemia. Also vitamin K enhances the activation of AMPK which can help in improving quality of life in cancer patients.

### Insulin Sensitivity and NIDDM

Non-insulin-dependent diabetes mellitus (NIDDM), Type 2 diabetes, is a common disease in the urban areas, globally. It is a major cause of cardiovascular disease and all-cause mortality [105-109]. Its prevalence is on the rise continuously for the last few decades [109].

Data from several prospective studies show an inverse association between physical activity and diabetes [5]. Physical work capacity (VO₂ₘₐₓ) is strongly associated with insulin sensitivity of skeletal muscle and subcutaneous adipose tissue [110]. First-degree relatives of type 2 diabetic patients (offspring) are often characterized by insulin resistance and reduced physical fitness (VO₂ₘₐₓ) [111]. The role of lifestyle intervention in preventing development of Type 2 diabetes has been explored in several trials during recent years, and there is now a vast body of evidence demonstrating that physical activity often in combination with weight loss causes a considerable reduction in the risk of developing Type 2 diabetes [111].

Poor physical fitness is a strong indicator of an increased risk of developing diabetes. The cause of decreased VO₂ₘₐₓ in Type 2 diabetic subjects and their relatives has not yet been fully determined.

Inadequate tissue perfusion leads to the inadequate oxygen concentration within the tissues resulting in development of hypoxia. Matsumoto et al [112] in their review on disturbance of microcirculation due to unhealthy lifestyle, hypothesize that unhealthy lifestyle first causes disturbance of the microcirculation, prompting opening of arteriovenous shunts and increasing bypassing of blood. This prevents the delivery of glucose and insulin to cells of peripheral tissues, causing apparent reduction of insulin sensitivity. Disturbance of the microcirculation also causes oxidative stress in peripheral tissues by inducing ischemia and hypoxia. This oxidative stress is considered to further exacerbate reduction of insulin sensitivity.

O'Donnell et al. [113] in his study on the metabolic consequences of intermittent hypoxia in mice, shows that in lean, otherwise healthy mice, exposure to intermittent hypoxia produced whole-body insulin resistance and reduced glucose utilization in oxidative muscle fibers. They conclude that intermittent hypoxia can cause acute insulin resistance in otherwise lean healthy animals, and this response is not dependent on the activation of the autonomic nervous system.

Polotsky et al [114] and group in yet another study on genetically obese mice shows that obese mice exposed to intermittent hypoxia for 12 weeks (long term) developed a time dependent increase in fasting serum insulin levels and worsening glucose tolerance, consistent with an increase in insulin resistance.

Eriksson et al [115] studied non-diabetic 4637 men between the ages of 48 to 54. Physical fitness was measured in terms of lung vital capacity and oxygen uptake during ergometry. Those who developed NIDDM were found to have 16% lower mean estimated maximal oxygen uptake along with deterioration in other parameters. Similarly, Regensteiner et al [116], in a trial with controls showed that at maximal exercise, diabetic persons had a 24% lower maximal walking time and 20% lower maximal VO₂ than controls (both P<0.05), while hemodynamic measures did not differ between groups. During graded exercise, at work loads below the maximal one, the relationship between VO₂ and work load was significantly lower in persons with NIDDM than controls by an average of 16%. They suggest that limitations in oxygen delivery may impair exercise performance in otherwise healthy persons with diabetes [116].

In yet another trial with 255 patients over a period of 10 years a low maximal oxygen uptake (VO₂ₘₐₓ) was found in patients with NIDDM compared with sedentary control subjects. In 29% of the patients there were also higher blood pressure levels at rest and during exercise with impaired VO₂ₘₐₓ. At the end of the program the researchers conclude the importance of aerobic fitness for the patients with NIDDM [117].

The reduced rate of increase in oxygen consumption during increasing sub maximal work loads in NIDDM suggests that limitations in oxygen delivery may impair exercise performance in otherwise healthy persons with diabetes [116].

Persons with type II diabetes mellitus (DM), even without cardiovascular complications have decreased maximal oxygen consumption (VO₂ₘₐₓ) and sub maximal oxygen consumption (VO₂) during graded exercise compared with healthy controls [118].

Hyperinsulinimia and decrease in insulin sensitivity (insulin resistance) precedes NIDDM and even pre-diabetic conditions like Impaired Fasting Glucose (IFG) and Impaired Glucose Tolerance (IGT) in obesity, Polycystic Ovarian Syndrome (PCOS), menopause, aging, etc.

Prevalence of hyperinsulinimia and insulin resistance is 60-70% in women with PCOS. Metabolic Syndrome and/or insulin resistance are reported 16-50% in menopausal women [119, 120].

Women with PCOS have associated metabolic dysfunction like dyslipidimia, high triglycerides and low HDL, dysglycemia hypertension etc., due to impaired insulin action in presence of hyperinsulininemia. It has been suggested that though insulin's metabolic action is defective in these patients, their mitotic function is intact. This causes proliferative disease at various sites like capillaries, ovarian stroma, uterine endometrium and acanthosis nigricans (thickening and darkening of the skin).

Eriksson et al [121], in a 15-year follow-up study, concluded that Increased skeletal muscle capillary density precedes diabetes development in men with impaired glucose tolerance. They suggest that the increased circulating insulin concentrations in IGT (impaired glucose tolerance) subjects have a capillary proliferative effect, perhaps to compensate for reduced capillary insulin diffusion and metabolic capacity of the muscle. These results suggest that diffusion distance from capillary to muscle cells or some associated biochemical change, and fiber type, could play a role in determining in vivo insulin action [122].

Yoshida et al [123], in a 36-month, randomized, double-blind, controlled trial with vitamin K supplementation of 500 μg/day showed a reduction in progression of insulin resistance in older men. However, no attempt was made to study the differences in the energy levels of the control and the interventional groups despite the long duration of the study. Inventors have proposed that the activation of the AMPK, and down stream effects on insulin action such as AKT phosphorylation and Glut4 translocation play a role in the clinical demonstration of retarded progression of insulin resistance in the trial.

AMPK is considered as a master switch in regulating glucose and lipid metabolism and that, as claimed, vitamin K stimulates AMPK status.

Winder et al [124] states that the net effect of AMPK activation is stimulation of hepatic fatty acid oxidation and ketogenesis, inhibition of cholesterol synthesis, lipogenesis, and triglyceride synthesis, inhibition of adipocyte lipolysis and lipogenesis, stimulation of skeletal muscle fatty acid oxidation and muscle glucose uptake, and modulation of insulin secretion by pancreatic beta-cells. The aforesaid changes contribute to the induction of Non-Alcoholic Fatty Liver Disease (NAFLD). Inventors propose that AMPK activation by the vitamin K or its derivatives, alone or in combinations there of with other active agents alleviate the syndrome.

In skeletal muscle, AMPK is activated by contraction [124]. However, defects or disuse (due to a sedentary life style) of the AMPK signaling system would be predicted to result in many of the metabolic perturbations observed in Type 2 diabetes mellitus. They further mentioned that increased recruitment of the AMPK signaling system may be effective in correcting insulin resistance in patients with forms of impaired glucose tolerance and Type 2 diabetes resulting from defects in the insulin signaling cascade.

Misra et al [125] state that along with evidence from studies showing that chemical activation of AMPK in vivo with 5-aminoimidazole-4-carboxamide ribonucleoside (AICAR) improves blood glucose concentrations.

We claim that supplementation of vitamin K or its derivatives, alone or in combinations there of with other active agents to humans to reduce significant risk factor of the physical work capacity (VO₂ₘₐₓ) in diabetic patients and their first degree relatives. We claim that increased tissue perfusion and reduction of hypoxia and hypoxemia with supplementation of vitamin K, as claimed in applicant's earlier patent application PCT/IN2008/000465 incorporated herein by reference, enhances the activation of AMPK ameliorating progression of insulin resistance and NIDDM.

### Coronary Artery Disease (CAD)

Inadequate tissue perfusion causes impairment in microcirculation which leads to the deprivation of necessary nutrients and oxygen resulting in the damage to the myocardium and other parts of heart. This in turn increases all causes of morbidity and mortality.

Sanchez et al [126] state that “ . . . Maximal exercise performance in patients with chronic heart failure, as determined by peak oxygen consumption (VO₂ₘₐₓ) during exercise testing has been shown to correlate well with mortality.'.

Blair et al [12] conducted a large study, at the Cooper Institute for Aerobic Research, Dallas, wherein there were 25341 men and 7080 women participants who completed preventive medical examinations, including a maximal exercise test, with a view to determine influence of VO₂ₘₐₓ and other precursors on CVD and all-cause mortality in men and women. Their findings bring forth importance VO₂ₘₐₓ. Low VO₂ₘₐₓ is a predictor of premature mortality. Further publications, from the same study group reinforce earlier findings and add clarity 1. A maximal exercise test (VO₂ₘₐₓ) performed in asymptomatic men free of cardiovascular disease does appear to be a worthwhile tool in predicting future risk of CHD”[23] 2. Low cardiorespiratory fitness (low VO₂ₘₐₓ) and physical inactivity are independent predictors of all-cause mortality in men with type 2 diabetes [25].

Lakka et al [8], in their study, find that “good cardiorespiratory fitness (VO₂ₘₐₓ) is associated with slower progression of early atherosclerosis in middle-aged men”.

Information from these studies allowed further studies to be more focused and concluded that “Cardiorespiratory fitness (VO₂ₘₐₓ) had a strong, graded, inverse association with overall, CVD-related, and non-CVD-related mortality. Maximal oxygen uptake (VO₂ₘₐₓ) and exercise test duration represent the strongest predictors of mortality”. Weisman, researching at William Beaumont Army Medical Center, El Paso, Tex., comes to the same conclusion on the importance of VO_{2max [}127].

Sedentary life style i.e. low physical activity has a negative influence on blood pressure and cholesterol, and may lead to diabetes. The study conducted by University of Hong Kong [128] states that the sedentary lifestyle is more dangerous for health than smoking. They reported that the 20% of all deaths of people 35 yrs and older were attributed to a lack of physical activity. They further mentioned that the risk of dying from heart disease was 52% higher for men and 28% higher for women, all due to a lack of physical activity.

Vermeer [129], in his patent application claims use of vitamin K and its derivatives to recover elasticity of the age-related stiffening of the arteries. His claim is for the useful role of vitamin K in reducing and reversing medial calcification of coronary artery and subsequent improvement in myocardial perfusion. To achieve this, Vermeer claims the treatment period lasting 6 months to 36 months with the ideal period as 36 months. Inventors of the current patent application do not claim their novel discovery based on the decalcification and nor do they wait for the decalcification. Inventors claim early benefits within a much shorter period of 30 days to 6 months and optimally a period of 2 months.

Also, the energy transduction improvement claimed in the current invention at the mitochondrial level is a novel, non-obvious and very useful in a coronary artery disease, improving myocardial inotropic action.

Invention is additionally enhanced by the use of the novel derivatives and combinations which synergize the energy transduction as well as myocardial cell contractibility. The latter would increase ionotropism and myocardial ejection function.

Li et al [130] in their review article have mentioned that adiponectin activates AMPK phosphorylation and then promotes various ATP-generating pathways in heart. They also stated that phosphorylation of AMPK induced by adiponectin inhibits myocardial protein synthesis, and would diminish the pathological cardiac hypertrophy. AMPK activation also has a cardioprotective role against myocardial injury and apoptosis in the ischemic heart. Son et al [131] demonstrated that adiponectin also antagonizes the stimulatory effect of TNF-alpha on the vascular damage by restoration of the AMPK-dependent Gas6-mediated survival pathway. This study clearly shows that the AMPK activation has a significant role in antagonizing chronic inflammation and hence has anti-atherosclerotic effects.

Hence, as mentioned above, inventors are claiming that the result of improving VO₂ₘₐₓ and enhancing AMPK activity i.e. enhancing the ability to consume more oxygen and to improve the energy production, and ability to supply more oxygen to the cells results in the amelioration of the coronary artery diseases.

### Essential Hypertension

Essential hypertension is pandemic throughout the world. There is marked increase in risk of developing numerous cardiovascular and respiratory complications with hypertension.

Current treatment modalities emphasize the role of non-pharmacological interventions and physical activity besides a wide spectrum of drugs. Many studies [7, 132-142] have firmly established that the regular exercise reduces the incidence of hypertension [7, 132-142].

Karch et al [143] evaluated the coronary capillary density by histological examination of the myocardium in four types of patients: control individuals, patients with ischaemic heart disease, inflammatory heart disease, or a dilated cardiomyopathy. Using computerized image analysis, precise modelling of the role of each capillary within a tissue unit is now possible. The tissue areas perfused by each capillary thus overlap in a healthy individual, thereby ensuring optimum oxygenation and nutrition of the tissues. In contrast, during ischaemic, inflammatory or dilated heart disease and during hypertension, capillaries are separated by greater distances, and the tissue area dependent on each capillary unit is greater so that certain areas of the myocardium are perfused poorly or not at all.

Hagberg. et al [135] in his study on the effect of exercise training on essential hypertension, has shown that endurance exercise training reduces systolic and diastolic blood pressures in approximately 75% of people who have essential hypertension. These reductions were approximately 10 mmHg for both systolic and diastolic blood pressures.

Iwane et al [137] investigated the effect of walking 10,000 steps/day or more on blood pressure in mild essential hypertensive patients, had found that, irrespective of exercise intensity or duration walking 10,000 steps/day or more was effective in lowering blood pressure in hypertensive patients and VO₂ₘₐₓ rose significantly.

Sawada et al [7] in his five years prospective study for finding out the relation between physical fitness (VO₂ₘₐₓ) and incidence of hypertension in cohort of 16,525 out of which 425 subject were diagnosed as hypertensive in the fifth year, shows that low VO₂ₘₐₓ level is related to the higher incidence of hypertension.

Mughal et al [141] studied the effect of aerobic exercise on changes in blood pressure and VO₂ₘₐₓ. They concluded that brisk walking yielded significant increase in VO₂ₘₐₓ (P<0.05).

Quinn et al [142] studied the effect of mild to moderate acute, endurance exercise in hypertensive individuals. They found that an exercise bout conducted between 50-75% VO₂ₘₐₓ significantly decreases systolic blood pressure and diastolic blood pressure in hypertensive subjects and that a greater and longer-lasting absolute reduction for 11 hrs after 75% bout compared to 4 hrs after 50% intensity.

Atherosclerosis is the basic lesion aggravating hypertension and its complications. Further atherosclerosis can impair the ability of skeletal muscle to utilize O₂ i.e. affecting cardiovascular fitness. In atherosclerosis elevated C-reactive protein (CRP) is a major risk factor. Kuo et at [139] examined 1438 adults aged 20-49 years for C-reactive protein (CRP) and VO₂ₘₐₓ. They found that CRP levels, inversely related to VO₂ₘₐₓ. Both lower CRP and higher VO₂ₘₐₓ are important indicators of exercise tolerance and cardiorespiratory fitness. Available literature, including patent literature [129] suggests calcification as a putative risk factor for cardiovascular disease as well as for hypertension. However the available data, by vitamin K supplementation, and animal models and other wise is for prevention of calcification but not for reverse. Hence reversal of calcification is postulated as a mechanism for lowering hypertension. These inventors have not claimed that lowering of CRP and improvement in VO₂ₘₐₓ are the activities of vitamin K. Unlike that, the present inventors have proposed a novel mechanism with the effect of vitamin K on energy transduction with cardiovascular protective action against diverse risk factors in hypertension and coronary artery disease.

Here the inventors claim that use of vitamin K or its derivatives, alone or in combinations thereof with other active agents and vitamin K based formulations improves tissue perfusion, improvement in VO₂ₘₐₓ, and enhances the activation of AMPK.

The applicants, in the current disclosure claim supplementation of vitamin K or its derivatives, alone or in combinations there of with other active agents and vitamin K containing formulation reduces inflammatory marker CRP and consequences thereof.

### DEFINITIONS

As used herein, the term “AMP” refers to adenosine monophosphate.

As used herein, the term “ADP” refers to adenosine diphosphate.

As used herein, the term “AMPK” refers to AMP-activated protein kinase

As used herein, the term “ATP” refers to adenosine triphosphate.

As used herein, the term “BMI” refer to Body Mass Index.

As used herein, the term cytochromes are, in general, membrane-bound hemoproteins that contain heme groups and carry out electron transport.

As used herein, the term “CVD” refer to Cardio Vascular Disease.

As used herein, the term “ETC” refers to Electron Transport Chain.

As used herein, the term “Hypoxia” is a pathological condition in which the body as a whole (generalized hypoxia) or region of the body (tissue hypoxia) is deprived of adequate oxygen supply.

As used herein, the term “Hypoxemia” is the reduction of oxygen content specifically in the arterial blood.

As used herein, the term “LSRD” refer to Life-Style Related Diseases (LSRD).

As used herein, the term “MONW” refer to Metabolically Obese, Normal individuals.

As used herein, the term “NAD” refers to Nicotinamide adenine dinucleotide,

As used herein, the term “NIDDM” refer to Non-Insulin-Dependent Diabetes Mellitus.

As used herein, the term pO₂ (Partial Pressure of Oxygen) reflects the amount of oxygen gas dissolved in the blood.

VO₂ₘₐₓ is “the highest rate of oxygen consumption attainable during maximal or exhaustive exercise”

As used herein, the term “vitamin K” refers to any molecule having vitamin K activity whether natural, e.g. PK, MK-n, or synthetically derived or analogues, derivatives or vitamin K-like compound.

As used herein, combination of vitamin K with “other active agents” includes but not restricted to combinations with synthetic molecules, natural product derivatives and medicinal plants. An exemplary list of active agents is provided in Table 1 with vitamin K or its derivatives.

As used herein, the term “PK” refers to phylloquinone (K1) or vitamin K1, also known as phytonadione because of its relationship with photosynthesis.

As used herein, the term “MK-n” refers to Menaquinones (K2) or vitamin K2, abbreviated as “MK-n.” The “n” signifies the number of unsaturated isoprene units that compose the side chain at the 3-position which may vary between 1 and 14.

As used herein, the term “K3” refers to menadione or vitamin K3.

As used herein, the term vitamin K and its derivatives refer to vitamin K and its analogues like vitamin K1 (PK), vitamin K2 (MK), esp. vitamin K2-4 (MK-4), vitamin K2-7 (MK-7), and vitamin K3 (K3), natural or synthetic, and their derivatives.

As used herein, the term “active sports” refers to all sports including but not restricted to soccer and tennis.

Therapeutically effective active agents as used herein means, those agents that are therapeutically effective and conventionally or unconventionally used for diseases or conditions encompassed by the current invention.

By restoration of energy, it is meant that the energy level in a mammalian subject is partially or completely restored.

Vitamin K and its derivatives play a fundamental role in diverse disease conditions. Table 1 enumerates various ingredients in different indications. Inventors claim that these ingredients have synergistic effect when administered in combination with vitamin K in each of the indications provided in Table 1. In addition, many more “active agents” that could potentially bring synergistic action when combined with vitamin K and its derivatives are encompassed by this invention.

TABLE 1
Exemplary Agents that provide synergistic effect
when combined with vitamin K and its derivatives

| | Synthetic | Natural Product | |
|---|---|---|---|
| Indication | Molecules | Derivatives | Medicinal Plants |
| Insulin Resistance/ | Metformin | Curcumin | 1. Phyllanthus emblica. |
| Metabolic Syndrome | | | 2. Mordica Charantia |
| Diabesity | Thiazolidine Dioses | Swartzia Marine | 1. Guar gum |
| | | | 2. Eugenia jambolana. |
| | | | 3. Gymnema Sylvestre |
| Hyperlipidemia & | Statins | Guggulsterones | 1. Terminalia chebula Retz. |
| Atherosclerosis | | Red rice yeast | 2. Allium Sativum |
| | | Policosanol | 3. Psyllium Seeds |
| Sports & Athletics | L-carnitine, L- | Nor-camphane | Siberian Ginsing |
| | Arginine | CoQ 10 | |
| | | Resveratrol | |
| NAFLD | Methyl Sulphonyl | Andrographolides | 1. Curcuruma longa Linn. |
| | Methane (MSM) | Glycyrrhizin | 2. Zingiber officinale |
| | Betaine | | |
| | Choline | | |
| Hypertension | Ace-inhibitors | Reserpine | 1. Nardostachys Jatamansi |
| | β-blocker | | 2. Boerhaavia diffusa |
| | Anagiotensin II | | |
| | antagonists | | |
| Coronary Heart | Nitroglycerin | Vitamin D group | 1. Terminalia Arjuna. |
| Diseases | ISDN | | 2. Picrorhiza kurroa |
| | Digoxin | | |
| | β-blockers | | |
| Muscle weakness | Creatin-phoshate | | Withania Somnifera |
| Anti-Aging: | | | |
| 1. Cognitive | Piracetam | Gingkolides | 1. Centella asiatica (Linn) |
| Decline | | Vincamine | 2. Butea fromdosa |
| 2. Parkinson | L-dopa | | 1. Mucuna puriens |
| Disease | Bromo-ergocryptine | | 2. Zizyphus jujuba |
| 3. Alzheimer's | Rivastigmine | Galantamine | 1. Salvia officinalis |
| Disease. | | | 2. Melissa officinalis |
| Cancer | Cancer Chemo | Taxol | 1. Withania Somnifera |
| | therapeutic agents | Vincristine | 2. Azadirachta indica |
| | | Vinblastine | |
| Peripheral | Vitamin B12, Folic | Capsicin, | 1. Cannabis India |
| Neuropathy | acid, Betaine, MSM | | |
| Muscle cramps | Baclofen, Calcium | Quinin | |
| | fructoborate | | |
| Chronic venous | Glyvenol | | Horse chest nut |
| insufficiency | | | seed extract |
| AMPK activator | AICAR, | Berbarine | Camellia sinensis |
| | Resveratrol, | | |
| | Theophyline, | | |

### EXAMPLES

### Example 01

Male, 32 years, with high moderate fitness, kept on mix of cardiovascular and resistance training exercise for 3-5 hrs per week, along with 3 capsules per day of MyoMax™ containing 450 μg of vitamin K2-7, for 2 weeks. Capsule was taken p.o. after baseline VO₂ₘₐₓ done which was 44.4 ml/kg/min. Within 2 weeks his VO₂ₘₐₓ changed from 44.4 ml/kg/min to 53.5 ml/kg/min. The observed percentage of increment was about 17%.

### Example 02

Male, 41 years, with High moderate fitness, kept on exercise for less than 3 hrs per week, for 2 weeks, along with 6 capsules per day of MyoMax™ containing 750 μg of vitamin K2-7, taken just for 4 days. Capsule was taken p.o. after baseline VO₂ₘₐₓ done which were 48.6 ml/kg/min. His VO₂ₘₐₓ changes from 48.6 ml/kg/min to 82.0 ml/kg/min within 2 weeks. The observed percentage of increment was about 41%.

### Example 03

Male, aged 68 years with hypercholesterolemia (Lipid Profile; 200 mg—Total cholesterol) for which he is taking Atorvastatin 5 mg one per day and 75 mg of Aspirin one per/day since last five years. At the age of 64 underwent angioplasty. He was also having blood pressure which ranges from 136-134/90-88 mmHg. He was taking Telmisartan 40 mg/day from last 4 years for hypertension. Then about 6 months ago he started taking vitamin K2-7 in daily dose of 200 μg. Currently he has stopped taking anti-hypertensive medication from last 1 month. Over a period of 1 and ½ months it is observed that his blood pressure has dropped down to 118-114/78-84 mmHg. Now he is off any anti-hypertensive medicine and continues with vitamin K2-7 only.

### Example 04

Female, aged 50 years suffering from general fatigue. She used to go to Gold gym 5-6 times per month. On the treadmill she used to walk at 5 km/hr speed at the start of the exercise and then gradually increase it to 6 km/hr in the next 15 minutes. When she started taking vitamin K2-7 350 μg per day, within 2 weeks of taking vitamin K2-7, she could start at 6 km/hr and go on up to 6.4 km/hr on the treadmill without feeling the strain she felt earlier. Her general fatigue had reduced. Her treadmill experience, was now smooth and ‘effortless’.

### Example 05

Woman aged 63, was diagnosed to have bilateral ovarian cancer in 2005, with ascites and abdominal lymph node metastases. She has undergone surgery and several courses of chemotherapy. Despite that she had a recurrence in June 2007 and subsequently lesions in liver in June 2008. She is currently under chemotherapy cycles. She tolerated chemotherapy reasonably well but complained of marked fatigue and low energy levels. She was started with vitamin K2-7, 100 μg. per day which gave her not much relief in fatigue and energy level. She was given a larger dose of vitamin K2-7 per day. Within six weeks, she reported a sense of well-being, less fatigue and significant improvement in the energy level. She is continued on the dose of vitamin K2-7 for two months. Her complaint of post-chemo restlessness/akathisia did not benefit from vitamin K2-7, but was relieved by phenergan. Her CEA levels have dropped markedly.

### Example 06

Male aged 72, had a history of subendocardial infarct (15 years back), hypertension and reasonably controlled moderate diabetes mellitus. His complaints were fatigue, low energy level, muscle cramps and tingling and numbness of lower limbs. The symptoms of the nerves and muscle were significantly relieved with vitamin K2-7 100 μg per day. These returned with drawal and were relieved with re-therapy. However the fatigue and low energy levels were only mildly relieved. He was started 350 μg per day of vitamin K2-7 over the two months period. He (a physician) has noted significant relief in fatigue and reasonable increases in the energy level. His cramps and neuropathic symptoms continue to be markedly reduced. There were no side effects with MK-7.

### Example 07

Male aged 56 yrs was having hypertension (160/114 mmHg) but was not on any medication. He complained of fatigue and low energy level. He was put on 3 capsules per day of 100 μg of vitamin K2-7 for 1 month and then on 2 capsule per day of 350 μg of vitamin K2-7 for the next 2 months under close observation by medical doctor. He showed decrease in systolic blood pressure (140/108 mmHg). He continued the medicine for next couple of month with same dose i.e. 2 capsules per day of 350 μg of vitamin K2-7. The blood pressure showed further drop to 138/104 mmHg. Then he was taken off from the treatment for 15 days. Blood pressure shot up to 146/108 mmHg, He was again put on vitamin K2-7, 350 μg, 2 capsules per day and his blood pressure came down to 136/100 mmHg. He also feels improvement in fatigue and feels more energetic at work.

### Example 08

Male aged 49, healthy otherwise, was having blood pressure on higher side i.e. 130/90 mmHg. He was advised to start taking vitamin K2-7, in a dose of 650 μg, one capsule per day. After 2 months of drug intake, blood pressure showed marked reduction to 112/80 mmHg. There were no adverse effects noted in the period of the treatment.

### Example 09

Male aged 56 yrs, was heaving history of Spondylosis, Diabetes mellitus and Hypertension. Five years ago he was operated for cataract (right eye). He is on regular medicine i.e. Atenol50™, one per day, after breakfast, Insulin2™ one per day after lunch, and Diaonil5™, two times a day. He was complaining of fatigue and lack of energy so much so that he found ascending 20 steps at a time difficult with related exhaustion and breathlessness. He was advised to take vitamin K2-7, 350 μg, one cap per day. After a month of time he started feeling more energetic and would walk up the stairs easily without any problem.

### Example 10

Four participants of age ranging from 32 to 40 yrs of both the sex were enrolled in a study where change in VO₂ₘₐₓ was studied with subsequent change in VO₂, upon administration of vitamin K2-7. All the participants were trained athletes and so any change in VO₂ was very interesting as the better trained one was, the harder it was to increase VO₂ₘₐₓ. VO₂ was measured at AB (Aerobic Base) and at AT (Anaerobic Threshold). VO₂ₘₐₓ was measured at AT (Anaerobic Threshold). All patients were given 600 μg per day, 300 μg in the morning and 300 μg in the evening of vitamin K2-7 for 21 days.

Table 2 shows the demographic data of participants.

TABLE 2
Demographic data

| Sr. No. | Patient ID | Sex/Age | BMI |
|---|---|---|---|
| 1 | TZ | M/40 | 25.1 |
| 2 | SR | F/40 | 24.2 |
| 3 | KL | F/32 | 20.8 |
| 4 | KK | F/33 | 27.4 |

After 21 days of treatment there were significant increases of VO₂ at AB when compared to VO₂ₘₐₓ. There was significant increase of oxygen carrying at AB and AT. Also, heart rates at AB and AT increased significantly as well. An overall increase in AB, AT and VO₂ₘₐₓ shows a significant increase in cardiovascular fitness.

As shown in Table 3 (a) and (b) there was average increase in VO₂ at AB was 8 mL/Kg/min and VO₂ at AT was 6.75 mL/Kg/min as compared to baseline.

From an athletic performance standpoint, VO₂ at AB and AT are actually more important as work (in watts) was done at AB and AT heart rates. The more VO₂ at AB and AT, the more watts of work one can perform at those points.

AB is where one is at the optimum aerobic/mitochondrial respiration. Body uses O₂ as the primary electron acceptor and burning fat for energy. The higher the AB, the faster, longer and harder work can be performed. Increasing VO₂ at AB is important and signifies improvement in cardiovascular fitness.

AT is where the body begins to switch to mostly anaerobic respiration. The higher the VO₂ at AT the more work can be done and the longer one can sustain that work at their AT. In Table 4, VO₂ₘₐₓ shows increasing trend from baseline. The average increase at this point is of 5 mL/Kg/min, which is significant. This was again very interesting finding as any improvement in VO_{2 max} translates to improvement in cardiovascular fitness.

TABLE 3
(a): VO₂ at AB

| | Pre (VO₂AB) | Post (VO₂AB) | |
|---|---|---|---|
| Subject | mL/Kg/min | mL/Kg/min | Change |
| TZ | 37 | 45 | 8 |
| SR | 30 | 36 | 6 |
| KL | 25 | 30 | 5 |
| KK | 28 | 41 | 13 |
| Avg | 30 | 38 | 8 |

(b): VO₂ at AT

| | Pre (VO₂AT) | Post (VO₂AT) | |
|---|---|---|---|
| Subject | mL/Kg/min | mL/Kg/min | Change |
| TZ | 44 | 49 | 5 |
| SR | 37 | 42 | 5 |
| KL | 42 | 42 | 0 |
| KK | 34 | 51 | 17 |
| Avg | 39.25 | 46 | 6.75 |

TABLE 4
VO_{2 max} at AT

| | Pre (VO₂ₘₐₓ) | Post (VO₂ₘₐₓ) | |
|---|---|---|---|
| Subject | mL/Kg/min | mL/Kg/min | Change |
| TZ | 53 | 54 | 1 |
| SR | 43 | 46 | 3 |
| KL | 46 | 49 | 3 |
| KK | 41 | 54 | 13 |
| Avg | 45.75 | 50.75 | 5 |

Table 5 (a) and (b) show the improvement in beats per minute (bpm) at AB and AT after treatment with vitamin K2-7.

Overall increase in VO₂ also increases AB and AT heart rate. Increasing AB and AT is a milestone in cardiovascular training for athletes. For non-athletes, increasing AB and AT is a mark of increased cardiovascular fitness.

Vitamin K2-7 appears to achieve this by the hypothesized mechanism proposed earlier. These results indicate that there was not only improvement in maximum oxygen utilization but also in the utilization of oxygen at both aerobic base and at aerobic threshold. There was also a marked improvement in the AB and AT heart rate. This all findings points toward the novel mechanism of action of vitamin K2-7 in the cardiovascular fitness.

TABLE 5
(a): Beats Per Minute (BPM) at AB

| | Pre | Post | |
|---|---|---|---|
| Subject | AB (bpm) | AB(bpm) | Change |
| TZ | 170 | 179 | 9 |
| SR | 160 | 173 | 13 |
| KL | 122 | 137 | 15 |
| KK | 144 | 177 | 33 |
| Avg | 149 | 166.5 | 17.5 |

(b): Beats Per Minute (BPM) at AT

| | Pre | Post | |
|---|---|---|---|
| Subject | AT (bpm) | AT(bpm) | Change |
| TZ | 178 | 187 | 9 |
| SR | 171 | 178 | 7 |
| KL | 162 | 163 | 1 |
| KK | 162 | 186 | 24 |
| Avg | 168.25 | 178.5 | 10.25 |

### REFERENCES

1. Wilmore J H, Costill D L: Physiology of Sport and Exercise. 3rd Edition edition. Champaign, Ill.: Human Kinetics 2005.
2. Farrand S K, Taber H W: Physiological effects of menaquinone deficiency in Bacillus subtilis. J Bacteriol 1973, 115(3):1035-1044.
3. Farrand S K, Taber H W: Changes in menaquinone concentration during growth and early sporulation in Bacillus subtilis. J Bacteriol 1974, 117(1):324-326.
4. Escamilla J E, Barquera B, Ramirez R, Garcia-Horsman A, del Arenal P: Role of menaquinone in inactivation and activation of the Bacillus cereus forespore respiratory system. J Bacteriol 1988, 170(12):5908-5912.
5. Wei M, Gibbons L W, Mitchell T L, Kampert J B, Lee C D, Blair S N: The association between cardiorespiratory fitness and impaired fasting glucose and type 2 diabetes mellitus in men. Ann Intern Med 1999, 130(2):89-96.
6. Sawada S S, Lee I M, Muto T, Matuszaki K, Blair S N: Cardiorespiratory fitness and the incidence of type 2 diabetes: prospective study of Japanese men. Diabetes Care 2003, 26(10):2918-2922.
7. Sawada S, Tanaka H, Funakoshi M, Shindo M, Kono S, Ishiko T: Five year prospective study on blood pressure and maximal oxygen uptake. Clin Exp Pharmacol Physiol 1993, 20(7-8):483-487.
8. Lakka T A, Laukkanen J A, Rauramaa R, Salonen R, Lakka H M, Kaplan G A, Salonen J T: Cardiorespiratory fitness and the progression of carotid atherosclerosis in middle-aged men. Ann Intern Med 2001, 134(1):12-20.
9. Wei M, Kampert J B, Barlow C E, Nichaman M Z, Gibbons L W, Paffenbarger R S, Jr., Blair S N: Relationship between low cardiorespiratory fitness and mortality in normal-weight, overweight, and obese men. JAMA 1999, 282(16):1547-1553.
10. Myers J, Prakash M, Froelicher V, Do D, Partington S, Atwood J E: Exercise capacity and mortality among men referred for exercise testing. N Engl J Med 2002, 346(11):793-801.
11. Laukkanen J A, Lakka T A, Rauramaa R, Kuhanen R, Venalainen J M, Salonen R, Salonen J T: Cardiovascular fitness as a predictor of mortality in men. Arch Intern Med 2001, 161(6):825-831.
12. Blair S N, Kampert J B, Kohl H W, 3rd, Barlow C E, Macera C A, Paffenbarger R S, Jr., Gibbons L W: Influences of cardiorespiratory fitness and other precursors on cardiovascular disease and all-cause mortality in men and women. JAMA 1996, 276(3):205-210.
13. Ottawa charter for health promotion. Can J Public Health 1986, 77(6):425-430.
14. Robertson A, Minkler M: New health promotion movement: a critical examination. Health Educ Q 1994, 21(3):295-312.
15. Nozaki S, Itokawa Y, Saito K, Sakurai H, Hirohata T: New Hygiene and Public Health. Edited by Administration LH. 370-1, Nankoudo, Tokyo; 2000.
16. Blair S N, Cheng Y, Holder J S: Is physical activity or physical fitness more important in defining health benefits? Med Sci Sports Exerc 2001, 33(6 Suppl):S379-399; discussion S419-320.
17. Blair S N, Kohl H W, 3rd, Paffenbarger R S, Jr., Clark D G, Cooper K H, Gibbons L W: Physical fitness and all-cause mortality. A prospective study of healthy men and women. JAMA 1989, 262(17):2395-2401.
18. Kawabata K, Imaki M, Ohguri M, Kondo H, Hayashi Y, Tanada S: [Study on the relationship between lifestyles and maximal oxygen uptake in healthy adults]. Nippon Eiseigaku Zasshi 1997, 52(2):470-474.
19. Kokkinos P F, Fernhall B: Physical activity and high density lipoprotein cholesterol levels: what is the relationship? Sports Med 1999, 28(5):307-314.
20. Montoye H J, Gayle R, Higgins M: Smoking habits, alcohol consumption and maximal oxygen uptake. Med Sci Sports Exerc 1980, 12(5):316-321.
21. Shephard R J: Absolute versus relative intensity of physical activity in a dose-response context. Med Sci Sports Exerc 2001, 33(6 Suppl):S400-418; discussion S419-420.
22. Tobita Y, Otaki H, Kusaka Y, lid M, Kajita E, Sato K: [A cross-sectional analysis on relationships between maximum oxygen uptake and risk factors for cardiovascular diseases]. Sangyo Eiseigaku Zasshi 1995, 37(6):409-415.
23. Gibbons L W, Mitchell T L, Wei M, Blair S N, Cooper K H: Maximal exercise test as a predictor of risk for mortality from coronary heart disease in asymptomatic men. Am J Cardiol 2000, 86(1):53-58.
24. Podolec P, Rubis P: [Heart failure and physical activity]. Przegl Lek 2007, 64(2):86-90.
25. Wei M, Gibbons L W, Kampert J B, Nichaman M Z, Blair S N: Low cardiorespiratory fitness and physical inactivity as predictors of mortality in men with type 2 diabetes. Ann Intern Med 2000, 132(8):605-611.
26. SHIHOKO S: Exercise and Physical Activity Reference Quantity 2006 (EPARQ2006). Japanese Journal of Nutrition and Dietetics 2006, 64(5):253-260.
27. Jackson A S, Beard E F, Wier L T, Ross R M, Stuteville J E, Blair S N: Changes in aerobic power of men, ages 25-70 yr. Med Sci Sports Exerc 1995, 27(1):113-120.
28. Jackson A S, Wier L T, Ayers G W, Beard E F, Stuteville J E, Blair S N: Changes in aerobic power of women, ages 20-64 yr. Med Sci Sports Exerc 1996, 28(7):884-891.
29. Narkar V A, Downes M, Yu R T, Embler E, Wang Y X, Banayo E, Mihaylova M M, Nelson M C, Zou Y, Juguilon H et al: AMPK and PPARdelta agonists are exercise mimetics. Cell 2008, 134(3):405-415.
30. ‘Exercise pill’ switches on gene that tells cells to burn fat [http://www.eurekalert.org/pub_releases/2007-04/foas-ps042107.php]
31. Armstrong L E, Casa D J, Roti M W, Lee E C, Craig S A, Sutherland J W, Fiala K A, Maresh C M: Influence of betaine consumption on strenuous running and sprinting in a hot environment. J Strength Cond Res 2008, 22(3):851-860.
32. Bredle D L, Stager J M, Brechue W F, Farber M O: Phosphate supplementation, cardiovascular function, and exercise performance in humans. J Appl Physiol 1988, 65(4):1821-1826.
33. Brown A C, Macrae H S, Turner N S: Tricarboxylic-acid-cycle intermediates and cycle endurance capacity. Int J Sport Nutr Exerc Metab 2004, 14(6):720-729.
34. Bryant R J, Ryder J, Martino P, Kim J, Craig B W: Effects of vitamin E and C supplementation either alone or in combination on exercise-induced lipid peroxidation in trained cyclists. J Strength Cond Res 2003, 17(4):792-800.
35. Bryner R W, Hornsby W G, Chetlin R, Ullrich I H, Yeater R A: Effect of lactate consumption on exercise performance. J Sports Med Phys Fitness 1998, 38(2):116-123.
36. Hsu C C, Ho M C, Lin L C, Su B, Hsu M C: American ginseng supplementation attenuates creatine kinase level induced by submaximal exercise in human beings. World J Gastroenterol 2005, 11(34):5327-5331.
37. Lee J K, Lee J S, Park H, Cha Y S, Yoon C S, Kim C K: Effect of L-carnitine supplementation and aerobic training on FABPc content and beta-HAD activity in human skeletal muscle. Eur J Appl Physiol 2007, 99(2):193-199.
38. McNaughton L, Dalton B, Tarr J: Inosine supplementation has no effect on aerobic or anaerobic cycling performance. Int J Sport Nutr 1999, 9(4):333-344.
39. Mero A, Raitanen R, Birkmayer J, Komi P: Effects of nicotinamide adenine dinucleotide hydride on physical and mental performance. J Sports Sci 2008, 26(3):311-319.
40. Misell L M, Lagomarcino N D, Schuster V, Kern M: Chronic medium-chain triacylglycerol consumption and endurance performance in trained runners. J Sports Med Phys Fitness 2001, 41(2):210-215.
41. Romer L M, Barrington J P, Jeukendrup A E: Effects of oral creatine supplementation on high intensity, intermittent exercise performance in competitive squash players. Int J Sports Med 2001, 22(8):546-552.
42. Syrotuik D G, Game A B, Gillies E M, Bell G J: Effects of creatine monohydrate supplementation during combined strength and high intensity rowing training on performance. Can J Appl Physiol 2001, 26(6):527-542.
43. U.S. Pat. No. 6,117,872, Maxwell A J, Cooke J P: Enhancement of exercise performance by augmenting endogenous nitric oxide production or activity, The Board of Trustees of the Leland Stanford Junior University (Stanford, Calif. 2000
44. U.S. Pat. No. 7,071,183, Montgomery H E, Martin J F, Erusalimsky J D: Use of inhibitors of the renin-angiotensin system., Ark Therapeutics Limited; 2006.
45. U.S. Pat. No. 6,368,640, Wuh H, Trant D W K: Method and composition for improving sexual fitness, The Daily Wellness Company; 2002.
46. Pollock M L: The quantification of endurance training programs. Exerc Sport Sci Rev 1973, 1:155-188.
47. Wilmore J H and Costill D L: Physiology of Sport and Exercise: 3rd Edition. Champaign, Ill.: Human Kinetics, 2005
48. Floreani M, Carpenedo F: One- and two-electron reduction of menadione in guinea-pig and rat cardiac tissue. Gen Pharmacol 1992, 23(4):757-762.
49. Thijssen H H, Vervoort L M, Schurgers L I, Shearer M J: Menadione i s a metabolite of oral vitamin K. Br J Nutr 2006, 95(2):260-266.
50. Goldenbaum P E, Keyser P D, White D C: Role of vitamin K2 in the organization and function of Staphylococcus aureua membranes. J Bacteriol 1975, 121(2):442-449.
51. Wang W, Fang H, Groom L, Cheng A, Zhang W, Liu J, Wang X, Li K, Han P, Zheng M et al: Superoxide flashes in single mitochondria. Cell 2008, 134(2):279-290.
52. Toyoda T, Hayashi T, Miyamoto L, Yonemitsu S, Nakano M, Tanaka S, Ebihara K, Masuzaki H, Hosoda K, Inoue G et al: Possible involvement of the alpha1 isoform of 5′AMP-activated protein kinase in oxidative stress-stimulated glucose transport in skeletal muscle. Am J Physiol Endocrinol Metab 2004, 287(1):E166-173.
53. Zhang M, Dong Y, Xu J, Xie Z, Wu Y, Song P, Guzman M, Wu J, Zou M H: Thromboxane receptor activates the AMP-activated protein kinase in vascular smooth muscle cells via hydrogen peroxide. Circ Res 2008, 102(3):328-337.
54. Stahmann N, Woods A, Carling D, Heller R: Thrombin activates AMP-activated protein kinase in endothelial cells via a pathway involving Ca2+/calmodulin-dependent protein kinase kinase beta. Mol Cell Biol 2006, 26(16):5933-5945.
55. Lee Y S, Kim W S, Kim K H, Yoon M J, Cho H J, Shen Y, Ye J M, Lee C H, Oh W K, Kim C T et al: Berberine, a natural plant product, activates AMP-activated protein kinase with beneficial metabolic effects in diabetic and insulin-resistant states. Diabetes 2006, 55(8):2256-2264.
56. Baur J A, Pearson K J, Price N L, Jamieson H A, Lerin C, Kalra A, Prabhu V V, Allard J S, Lopez-Lluch G, Lewis K et al: Resveratrol improves health and survival of mice on a high-calorie diet. Nature 2006, 444(7117):337-342.
57. Hwang J T, Ha J, Park I J, Lee S K, Baik H W, Kim Y M, Park O J: Apoptotic effect of EGCG in HT-29 colon cancer cells via AMPK signal pathway. Cancer Lett 2007, 247(1):115-121.
58. Hardie D G: AMP-activated/SNF1 protein kinases: conserved guardians of cellular energy. Nat Rev Mol Cell Biol 2007, 8(10):774-785.
59. Mortensen S P, Damsgaard R, Dawson E A, Secher N H, Gonzalez-Alonso J: Restrictions in systemic and locomotor skeletal muscle perfusion, oxygen supply and VO2 during high-intensity whole-body exercise in humans. J Physiol 2008, 586(10):2621-2635.
60. Bangsbo J: The physiology of soccer—with special reference to intense intermittent exercise. Acta Physiol Scand Suppl 1994, 619:1-155.
61. Chamari K, Moussa-Chamari I, Boussaidi L, Hachana Y, Kaouech F, Wisloff U: Appropriate interpretation of aerobic capacity: allometric scaling in adult and young soccer players. Br J Sports Med 2005, 39(2):97-101.
62. Helgerud J, Engen L C, Wisloff U, Hoff J: Aerobic endurance training improves soccer performance. Med Sci Sports Exerc 2001, 33(11):1925-1931.
63. Hoff J, Wisloff U, Engen L C, Kemi O J, Helgerud J: Soccer specific aerobic endurance training. Br J Sports Med 2002, 36(3):218-221.
64. Kemi O J, Hoff J, Engen L C, Helgerud J, Wisloff U: Soccer specific testing of maximal oxygen uptake. J Sports Med Phys Fitness 2003, 43(2):139-144.
65. Fernandez J, Mendez-Villanueva A, Pluim B M: Intensity of tennis match play. Br J Sports Med 2006, 40(5):387-391; discussion 391.
66. Lees A: Science and the major racket sports: a review. J Sports Sci 2003, 21(9):707-732.
67. Roetert P E, Brown S W, Piorkowski P A: Fitness comparisons among three different levels of elite tennis players. J Strength Cond Res 1996, 10:139-143.
68. Girard O, Chevalier R, Leveque F, Micallef J P, Millet G P: Specific incremental field test for aerobic fitness in tennis. Br J Sports Med 2006, 40(9):791-796.
69. Girard O, Lattier G, Micallef J P, Millet G P: Changes in exercise characteristics, maximal voluntary contraction, and explosive strength during prolonged tennis playing. Br J Sports Med 2006, 40(6):521-526.
70. Konig D, Huonker M, Schmid A, Halle M, Berg A, Keul J: Cardiovascular, metabolic, and hormonal parameters in professional tennis players. Med Sci Sports Exerc 2001, 33(4):654-658.
71. Hayashi T, Hirshman M F, Kurth E J, Winder W W, Goodyear L J: Evidence for 5′AMP-activated protein kinase mediation of the effect of muscle contraction on glucose transport. Diabetes 1998, 47(8):1369-1373.
72. Holmes B F, Kurth-Kraczek E J, Winder W W: Chronic activation of 5′-AMP-activated protein kinase increases GLUT-4, hexokinase, and glycogen in muscle. J Appl Physiol 1999, 87(5):1990-1995.
73. Winder W W, Holmes B F, Rubink D S, Jensen E B, Chen M, Holloszy J O: Activation of AMP-activated protein kinase increases mitochondrial enzymes in skeletal muscle. J Appl Physiol 2000, 88(6):2219-2226.
74. American College of Sports Medicine Position Stand. Exercise and physical activity for older adults. Med Sci Sports Exerc 1998, 30(6):992-1008.
75. Paterson D H: Effects of ageing on the cardiorespiratory system. Can J Sport Sci 1992, 17(3):171-177.
76. Yeap B B: Are declining testosterone levels a major risk factor for ill-health in aging men? Int J Impot Res 2008.
77. Innes K E, Selfe T K, Taylor A G: Menopause, the metabolic syndrome, and mind-body therapies. Menopause 2008, 15(5):1005-1013.
78. Huonker M, Schmidt-Trucksass A, Heiss H W, Keul J: [Effects of physical training and age-induced structural and functional changes in cardiovascular system and skeletal muscles]. Z Gerontol Geriatr 2002, 35(2):151-156.
79. Ruderman N B, Berchtold P, Schneider S: Obesity-associated disorders in normal-weight individuals: some speculations. Int J Obes 1982, 6 Suppl 1:151-157.
80. Ruderman N, Chisholm D, Pi-Sunyer X, Schneider S: The metabolically obese, normal-weight individual revisited. Diabetes 1998, 47(5):699-713.
81. Executive Summary of The Third Report of The National Cholesterol Education Program (NCEP) Expert Panel on Detection, Evaluation, And Treatment of High Blood Cholesterol In Adults (Adult Treatment Panel III). JAMA 2001, 285(19):2486-2497.
82. St-Onge M P, Janssen I, Heymsfield S B: Metabolic syndrome in normal-weight Americans: new definition of the metabolically obese, normal-weight individual. Diabetes Care 2004, 27(9):2222-2228.
83. Haffner S M, Valdez R A, Hazuda H P, Mitchell B D, Morales P A, Stern M P: Prospective analysis of the insulin-resistance syndrome (syndrome X). Diabetes 1992, 41(6):715-722.
84. Isomaa B, Almgren P, Tuomi T, Forsen B, Lahti K, Nissen M, Taskinen M R, Groop L: Cardiovascular morbidity and mortality associated with the metabolic syndrome. Diabetes Care 2001, 24(4):683-689.
85. Trevisan M, Liu J, Bahsas F B, Menotti A: Syndrome X and mortality: a population-based study. Risk Factor and Life Expectancy Research Group. Am J Epidemiol 1998, 148(10):958-966.
86. Ford E S, Giles W H, Dietz W H: Prevalence of the metabolic syndrome among US adults: findings from the third National Health and Nutrition Examination Survey. JAMA 2002, 287(3):356-359.
87. Conus F, Allison D B, Rabasa-Lhoret R, St-Onge M, St-Pierre D H, Tremblay-Lebeau A, Poehlman E T: Metabolic and behavioral characteristics of metabolically obese but normal-weight women. J Clin Endocrinol Metab 2004, 89(10):5013-5020.
88. Bolliger C T, Jordan P, Soler M, Stulz P, Gradel E, Skarvan K, Elsasser S, Gonon M, Wyser C, Tamm M et al: Exercise capacity as a predictor of postoperative complications in lung resection candidates. A m J Respir Crit Care Med 1995, 151(5):1472-1480.
89. Wang J S: Pulmonary function tests in preoperative pulmonary evaluation. Respir Med 2004, 98(7):598-605.
90. Wang J S, Abboud R T, Evans K G, Finley R J, Graham B L: Role of CO diffusing capacity during exercise in the preoperative evaluation for lung resection. A m J Respir Crit Care Med 2000, 162(4 Pt 1):1435-1444.
91. Benzo R, Kelley G A, Recchi L, Hofman A, Sciurba F: Complications of lung resection and exercise capacity: a meta-analysis. Respir Med 2007, 101(8):1790-1797.
92. Gaballo A, Corbo G M, Valente S, Clappi G: Preoperative evaluation and risk factors of lung cancer. Rays 2004, 29(4):391-400.
93. Richard D E, Berra E, Pouyssegur J: Angiogenesis: how a tumor adapts to hypoxia. Biochem Biophys Res Commun 1999, 266(3):718-722.
94. Giatromanolaki A, Harris A L: Tumour hypoxia, hypoxia signaling pathways and hypoxia inducible factor expression in human cancer. Anticancer Res 2001, 21(6B):4317-4324.
95. Liotta L A, Steeg P S, Stetler-Stevenson W G: Cancer metastasis and angiogenesis: an imbalance of positive and negative regulation. Cell 1991, 64(2):327-336.
96. Hanahan D, Folkman J: Patterns and emerging mechanisms of the angiogenic switch during tumorigenesis. Cell 1996, 86(3):353-364.
97. Brizel D M, Scully S P, Harrelson J M, Layfield Li, Bean J M, Prosnitz L R, Dewhirst M W: Tumor oxygenation predicts for the likelihood of distant metastases in human soft tissue sarcoma. Cancer Res 1996, 56(5):941-943.
98. Hockel M, Schlenger K, Aral B, Mitze M, Schaffer U, Vaupel P: Association between tumor hypoxia and malignant progression in advanced cancer of the uterine cervix. Cancer Res 1996, 56(19):4509-4515.
99. Megibow A J: Pancreatic adenocarcinoma: designing the examination to evaluate the clinical questions. Radiology 1992, 183(2):297-303.
100. Evans W J: Physical function in men and women with cancer. Effects of anemia and conditioning. Oncology (Williston Park) 2002, 16(9 Suppl 10):109-115.
101. Schmitz K H, Holtzman J, Courneya K S, Masse L C, Duval S, Kane R: Controlled physical activity trials in cancer survivors: a systematic review and meta-analysis. Cancer Epidemiol Biomarkers Prey 2005, 14(7):1588-1595.
102. Sawada S S, Muto T, Tanaka H, Lee I M, Paffenbarger R S, Jr., Shindo M, Blair S N: Cardiorespiratory fitness and cancer mortality in Japanese men: a prospective study. Med Sci Sports Exerc 2003, 35(9):1546-1550.
103. Huang X, Wullschleger S, Shpiro N, McGuire V A, Sakamoto K, Woods Y L, McBurnie W, Fleming S, Alessi D R: Important role of the LKB1-AMPK pathway in suppressing tumorigenesis in PTEN-deficient mice. Biochem J 2008, 412(2):211-221.
104. Papandreou I, Lim A L, Laderoute K, Denko N C: Hypoxia signals autophagy in tumor cells via AMPK activity, independent of HIF-1, BNIP3, and BNIP3L. Cell Death Differ 2008, 15(10):1572-1581.
105. Klein R: Hyperglycemia and microvascular and macrovascular disease in diabetes. Diabetes Care 1995, 18(2):258-268.
106. Stamler J, Vaccaro O, Neaton J D, Wentworth D: Diabetes, other risk factors, and 12-yr cardiovascular mortality for men screened in the Multiple Risk Factor Intervention Trial. Diabetes Care 1993, 16(2):434-444.
107. Wei M, Mitchell B D, Haffner S M, Stern M P: Effects of cigarette smoking, diabetes, high cholesterol, and hypertension on all-cause mortality and cardiovascular disease mortality in Mexican Americans. The San Antonio Heart Study. Am J Epidemiol 1996, 144(11):1058-1065.
108. Lowe L P, Liu K, Greenland P, Metzger B E, Dyer A R, Stamler J: Diabetes, asymptomatic hyperglycemia, and 22-year mortality in black and white men. The Chicago Heart Association Detection Project in Industry Study. Diabetes Care 1997, 20(2):163-169.
109. Report of the Expert Committee on the Diagnosis and Classification of Diabetes Mellitus. Diabetes Care 1997, 20(7):1183-1197.
110. Nyholm B, Nielsen M F, Kristensen K, Nielsen S, Ostergard T, Pedersen S B, Christiansen T, Richelsen B, Jensen M D, Schmitz O: Evidence of increased visceral obesity and reduced physical fitness in healthy insulin-resistant first-degree relatives of type 2 diabetic patients. Eur J Endocrinol 2004, 150(2):207-214.
111. Ostergard T, Andersen J L, Nyholm B, Lund S, Nair K S; Saltin B, Schmitz O: Impact of exercise training on insulin sensitivity, physical fitness, and muscle oxidative capacity in first-degree relatives of type 2 diabetic patients. Am J Physiol Endocrinol Metab 2006, 290(5):E998-1005.
112. Matsumoto Y, Ohno H, Noguchi I, Kikuchi Y, Kurihara T: Disturbance of microcirculation due to unhealthy lifestyle: Cause of type 2 diabetes. Med Hypotheses 2006, 66(3):550-553.
113. O'Donnell C P: Metabolic consequences of intermittent hypoxia. Adv Exp Med Biol 2007, 618:41-49.
114. Polotsky V Y, Li J, Punjabi N M, Rubin A E, Smith P L, Schwartz A R, O'Donnell C P: Intermittent hypoxia increases insulin resistance in genetically obese mice. J Physiol 2003, 552(Pt 1):253-264.
115. Eriksson K F, Lindgarde F: Poor physical fitness, and impaired early insulin response but late hyperinsulinaemia, as predictors of NIDDM in middle-aged Swedish men. Diabetologia 1996, 39(5):573-579.
116. Regensteiner J G, Sippel J, McFarling E T, Wolfel E E, Hiatt W R: Effects of non-insulin-dependent diabetes on oxygen consumption during treadmill exercise. Med Sci Sports Exerc 1995, 27(6):875-881.
117. Schneider S H, Khachadurian A K, Amorosa L F, Clemow L, Ruderman N B: Ten-year experience with an exercise-based outpatient life-style modification program in the treatment of diabetes mellitus. Diabetes Care 1992, 15(11):1800-1810.
118. Regensteiner J G, Bauer T A, Reusch J E, Brandenburg S L, Sippel J M, Vogelsong A M, Smith S, Wolfel E E, Eckel R H, Hiatt W R: Abnormal oxygen uptake kinetic responses in women with type II diabetes mellitus. J Appl Physiol 1998, 85(1):310-317.
119. Vaidya R, Pandey S, Vaidya A D B: Polycystic Ovary Syndrome: Is it a Chronic Inflammatory Dasease? In Polycystic Ovary Syndrome. Edited by Mukherjee G G. New Delhi: Elsevier, a division of Reed Elsevier India Pvt. Ltd.; 2008.
120. Mittal S: Menopause: A Metabolic Syndrome. In Menopause. Edited by Vaidya R. New Delhi: Elsevier, a division of Reed Elsevier India Pvt. Ltd.; 2008.
121. Eriksson K F, Saltin B, Lindgarde F: Increased skeletal muscle capillary density precedes diabetes development in men with impaired glucose tolerance. A 15-year follow-up. Diabetes 1994, 43(6):805-808.
122. Lillioja S, Young A A, Culter C L, Ivy J L, Abbott W G, Zawadzki J K, Yki-Jarvinen H, Christin L, Secomb T W, Bogardus C: Skeletal muscle capillary density and fiber type are possible determinants of in vivo insulin resistance in man. J Clin Invest 1987, 80(2):415-424.
123. Yoshida M, Jacques P F, Meigs J B, Saltzman E, Shea M K, Gundberg C, Dawson-Hughes B, Dallal G, Booth S L: Effect of vitamin K supplementation on insulin resistance in older men and women. Diabetes Care 2008.
124. Winder W W, Hardie D G: AMP-activated protein kinase, a metabolic master switch: possible roles in type 2 diabetes. Am J Physiol 1999, 277(1 Pt 1):E1-10.
125. Misra P: AMP activated protein kinase: a next generation target for total metabolic control. Expert Opin Ther Targets 2008, 12(1):91-100.
126. Gomez Sanchez M A, Fernandez Vaquero A, Delgado Jimenez J, Tello Meneses R, Lopez Chicharro J, Hernandez Alfonso J: [The importance of determining oxygen consumption in the indications for a heart transplant]. Rev Esp Cardiol 1995, 48 Suppl 7:19-23.
127. Weisman I M: Cardiopulmonary exercise testing in the preoperative assessment for lung resection surgery. Semin Thorac Cardiovasc Surg 2001, 13(2):116-125.
128. Sedentary lifestyle causes more deaths than smoking, says study [http://www.newstarget.com/001547.html]
129. US20060166948, Vermeer: Compositions comprising vitamin k for treating or preventing age-related stiffening of arteries. VITAK BV; 2003.
130. Li L, Wu L L: [Effect of AMP-activated protein kinase on cardiovascular protection of adiponectin.]. Sheng Li Xue Bao 2007, 59(5):614-618.
131. Son B K, Akishita M: [Vascular calcification and anti-aging]. Clin Calcium 2008, 18(7):912-917.
132. Chiriac S, Dima-Cozma C, Georgescu T, Turcanu D, Pandele G I: [The beneficial effect of physical training in hypertension]. Rev Med Chir Soc Med Nat Iasi 2002, 107(2):258-263.
133. de Wild G M, Peeters M P, Hoefnagels W H, Oeseburg B, Binkhorst R A: Relative exercise intensity of long-distance marching (120 km in 4 days) in 153 subjects aged 69-87 years. Eur J Appl Physiol Occup Physiol 1997, 76(6):510-516.
134. Dengel D R, Hagberg J M, Pratley R E, Rogus E M, Goldberg A P: Improvements in blood pressure, glucose metabolism, and lipoprotein lipids after aerobic exercise plus weight loss in obese, hypertensive middle-aged men. Metabolism 1998, 47(9):1075-1082.
135. Hagberg J M, Brown M D: Does exercise training play a role in the treatment of essential hypertension? J Cardiovasc Risk 1995, 2(4):296-302.
136. Hamer M, Taylor A, Steptoe A: The effect of acute aerobic exercise on stress related blood pressure responses: a systematic review and meta-analysis. Biol Psychol 2006, 71(2):183-190.
137. Iwane M, Arita M, Tomimoto S, Satani O, Matsumoto M, Miyashita K, Nishio I: Walking 10,000 steps/day or more reduces blood pressure and sympathetic nerve activity in mild essential hypertension. Hypertens Res 2000, 23(6):573-580.
138. Kishida T, Inaba R, Iwata H: [Relationships between maximal oxygen uptake (VO2max) and physical activity, blood pressure and serum lipids]. Nippon Eiseigaku Zasshi 1997, 52(2):475-480.
139. Kuo H K, Yen C J, Chen J H, Yu Y H, Bean J F: Association of cardiorespiratory fitness and levels of C-reactive protein: data from the National Health and Nutrition Examination Survey 1999-2002. Int J Cardiol 2007, 114(1):28-33.
140. McMurray R G, Ainsworth B E, Harrell J S, Griggs T R, Williams O D: Is physical activity or aerobic power more influential on reducing cardiovascular disease risk factors? Med Sci Sports Exerc 1998, 30(10):1521-1529.
141. Mughal M A, Alvi I A, Akhund I A, Ansari A K: The effects of aerobic exercise training on resting blood pressure in hypertensive patients. J Pak Med Assoc 2001, 51(6):222-226.
142. Quinn T J: Twenty-four hour, ambulatory blood pressure responses following acute exercise: impact of exercise intensity. J Hum Hypertens 2000, 14(9):547-553.
143. Karch R, Neumann F, Ullrich R, Neumuller J, Podesser B K, Neumann M, Schreiner W: The spatial pattern of coronary capillaries in patients with dilated, ischemic, or inflammatory cardiomyopathy. Cardiovasc Pathol 2005, 14(3):135-144.

What is claimed is:

1. A method of alleviating hypoxemia and hypoxia, resulting in increased energy levels, said method comprising administering to a mammal a composition of vitamin K or its derivatives alone or in combination with other active agents.

2. The method of claim 1 wherein the vitamin K or its derivatives activate AMPK which is associated with (a) restoration of energy in chronic fatigue syndrome (b) countering the post-ischemic effects in heart, brain, muscles, liver and kidneys (c) increase in life-span in caloric restrictions (d) improved quality of life in patients with cancer (e) significant amelioration of metabolic syndrome (f) reduction in inflammatory cytokines and improved adipose endocrine harmony (g) relief of lipid deposition in arteries and liver and (h) increased tumor suppression.

3. A method of improving the tissue perfusion, increasing VO2max and activation of AMPK comprising administering to a mammal a composition of vitamin K or its derivatives alone or in combination with other active agents.

4. The method of claim 3 wherein the said method of increasing VO2max influences the technical and tactical performance in all active sports.

5. A method to maintain a balanced homeostatic state in geriatric population by improvement in tissue perfusion and activation of AMPK, said method comprising administering to the said population a composition of vitamin K or its derivatives, alone or in combination with other active agents.

6. A method of ameliorating the condition of MONW in a mammalian subject, the said method comprising administering a composition of vitamin K or its derivatives, alone or in combination with other active agents.

7. A method to improve the quality of life in cancer patients by activation of AMPK comprising administering vitamin K or its derivatives, alone or in combination with other active agents.

8. (canceled)

9. A method of lowering significant risk factors of the physical work capacity (VO₂ₘₐₓ) in diabetic patients and their first degree relatives, the said method comprising supplementing vitamin K or its derivatives, alone or in combination with other active agents.

10. (canceled)

11. A method to lower inflammatory C-reactive protein (CRP) marker, the said method comprising supplementing a composition of vitamin K or its derivatives, alone or in combination thereof with other active agents.

12. The method of claim 11, wherein the said reduction in inflammatory CRP marker is associated with reduction in atherosclerosis and thereby reduction in hypertension.

13. A method of reducing normoxic hypoxia and hypoxemic hypoxia, the said method comprising supplementing a composition of vitamin K or its derivatives, alone or in combination with other active agents.

14. A method of maintaining enhanced O₂ level and/or improving the O₂ affinity of the mitochondrial enzyme system for adequate generation of ATP molecules, the said method comprising supplementing a composition of vitamin K or its derivatives, alone or in combination thereof with other active agents.
